(19) United States
(12) Patent Application Publication
(10) Pub. No.: US 2024/0225419 A1
(43) Pub. Date: Jul. 11, 2024

(54) ENDOSCOPE VALVE DEVICES, SYSTEMS, AND METHODS

(71) Applicant: BOSTON SCIENTIFIC SCIMED, INC., MAPLE GROVE, MN (US)

(72) Inventor: Juan M. Alvarez Azpeitia, Framingham, MA (US)

(73) Assignee: BOSTON SCIENTIFIC SCIMED, INC., MAPLE GROVE, MN (US)

(21) Appl. No.: 18/403,819

(22) Filed: Jan. 4, 2024

Related U.S. Application Data
(60) Provisional application No. 63/437,264, filed on Jan. 5, 2023.

(51) Int. Cl. A61B 1/00; A61B 1/015

(52) U.S. Cl. CPC A61B 1/00068; A61B 1/015

(57) ABSTRACT

A valve assembly with improved sealing. The valve assembly may be configured for use as a suction valve assembly. The valve assembly includes a valve well, and a valve shaft movable with respect to the valve well to shift the valve assembly between off and on configurations. A cylindrical sealing member is circumferentially positioned about the valve shaft to seal around a transverse port extending transversely through the valve shaft and in fluid communication, via a channel within the valve shaft, with an axial port at an end of the valve shaft. The cylindrical seal member seals the transverse port of the valve shaft (and a transverse valve well port in fluid communication therewith) from fluid communication with the axial port of the valve shaft (and an axial valve well port in fluid communication therewith) when the valve shaft assembly is within the valve well.

### CROSS-REFERENCE TO RELATED APPLICATIONS

This application claims the benefit of priority under 35 U.S.C. § 119 to U.S. Provisional Application No. 63/437,264, filed Jan. 5, 2023, the entire disclosure of which is hereby incorporated by reference herein for all purposes.

### FIELD

The present disclosure relates generally to devices (including, without limitation, components and assemblies), systems, and methods for controlling flow of materials through a valve. In particular, the present disclosure relates to devices, systems, and methods for controlling flow of materials through an endoscope, and particularly through a valve of a valve assembly usable in a medical device such as an endoscope.

### BACKGROUND

Various devices with valve assemblies are known in the art for use during various medical procedures. For instance, materials may be supplied to an anatomical site (e.g., fluid may be supplied, such as for irrigation), and/or suctioned from the anatomical site (e.g., fluid or biological materials may be withdrawn from an anatomical site) during a medical procedure. A valve assembly may be used to control flow of such materials. An endoscope is a common medical device used to introduce or remove substances with respect to an anatomical site, and thus typically includes a valve assembly. Endoscopes typically have an insertion tube with a working channel via which substances (e.g., fluids such as gas or liquids) or devices or instruments or tools may be introduced, or substances may be removed or suctioned out. To control flow of substances through an endoscope, a fluid source, and/or a suction pump/vacuum source is fluidly coupled with the endoscope handle and the insertion tube via a valve assembly. The valve assembly typically has a valve well and a valve shaft shiftable within the valve well between an off position in which the valve assembly is in an off configuration, and an on position in which the valve assembly is in an on configuration. In the off configuration, the valve assembly blocks fluid communication between the fluid source/suction source and the working channel of the endoscope. When the valve assembly is shifted into an on configuration (typically by being depressed with respect to the handle), fluid communication between the fluid/suction source and the insertion tube of the endoscope is established to supply fluid and/or to apply suction/negative pressure to the insertion tube of the endoscope. Proper sealing of the ports, channels, lumens, etc., associated with such valves is important. For instance, with respect to suction valve assemblies, seals are provided along the ports and channels of the suction flow path to maintain adequate suction force when the suction valve is in an on position (fluidly coupling the suction source with the working channel), as well as to prevent unintended suction through the working channel when the suction valve in the off position. There remains a need for improvements to endoscope valves, such as suction valves and seals associated therewith.

### SUMMARY

This Summary is provided to introduce, in simplified form, a selection of concepts described in further detail below in the Detailed Description. This Summary is not intended to necessarily identify key features or essential features of the claimed subject matter, nor is it intended as an aid in determining the scope of the claimed subject matter. One of skill in the art will understand that each of the various aspects and features of the present disclosure may advantageously be used separately in some instances, or in combination with other aspects and features of the disclosure in other instances, whether or not described in this Summary. No limitation as to the scope of the claimed subject matter is intended by either the inclusion or non-inclusion of elements, components, or the like in this Summary.

In accordance with various principles of the present disclosure, a valve component for a medical valve assembly includes a valve shaft extendable through a valve well of a valve assembly and along an actuation axis for actuation movement along the actuation axis, and defining a valve-shaft-suction-source port extending transverse to the actuation axis, and a valve-shaft-suction-application port extending axially along the actuation axis and in fluid communication with the valve-shaft-suction-source port; and a cylindrical seal member extending circumferentially around the valve shaft and defining a suction-source communication port in fluid communication with the valve-shaft-suction-source port. In some aspects, the valve shaft is axially movable within a valve well between an off position in which the valve assembly is in an off configuration with the valve-shaft-suction-source port not in fluid communication with the valve-well-suction-source port, and an on position in which the valve assembly is in an on configuration with the valve-shaft-suction-source port in fluid communication with the valve-well-suction-source port.

Optionally, the cylindrical seal member includes circumferential seal elements extending circumferentially around the cylindrical seal member and radially outwardly from the outer surface of the cylindrical seal member proximal and distal to the circumferential seal elements. Optionally, the circumferential seal elements form seals proximal and distal to the valve-shaft-suction-source port and the valve-well-suction-source port to create a sealed space extending circumferentially around the valve shaft to seal fluid communication through the valve assembly between a suction source fluidly in communication with the valve-well-suction-source port and a suction application device fluidly in communication with the valve-well-suction-application port.

In some aspects, the valve shaft has a proximal end extending away from the valve well for actuation by a user, and a distal end extending within the valve well. Optionally, the valve assembly further includes a shaft seal member adjacent the distal end of the valve shaft. In some aspects, the shaft seal member seals between the valve-well-suction-source port and the valve-well-suction-application port.

Optionally, the cylindrical seal member defines one or more suction bleed passages axially along the cylindrical seal member between the valve shaft and the cylindrical seal member. Optionally, the one or more suction bleed passages are in fluid communication with air outside the valve assembly to allow air from outside the valve assembly to bleed a suction source in fluid communication with the valve-well-suction-source port when the suction source is running and the valve assembly is in the off configuration. Optionally, the valve shaft defines a bleed port therethrough in fluid communication with air outside the valve assembly and with the one or more bleed passages. Optionally, the one or more bleed passages extend axially through the cylindrical seal member, and the cylindrical seal member seals between the one or more bleed passages therethrough and the valve-well-suction-source port.

In accordance with various principles of the present disclosure, a valve shaft assembly for a valve assembly of a medical device includes a valve shaft having a proximal end configured for engagement by a user, a distal end, and a longitudinal axis extending therebetween, the valve shaft defining a transversely-extending port extending transverse to the longitudinal axis, and an axially-extending port extending axially along longitudinal actuation axis, with the transversely-extending port in fluid communication with the axially-extending port; and a flexible cylindrical seal member formed from a sealing material more flexible than the valve shaft, and extending circumferentially around the valve shaft and defining a transversely-extending port in fluid communication with the transversely-extending port in the valve shaft.

Optionally, the cylindrical seal member includes circumferential seal elements extending circumferentially around the cylindrical seal member and radially outwardly from the outer surface of the cylindrical seal member proximal and distal to the circumferential seal elements. Optionally, the circumferential seal elements are configured to define seals proximal and distal to the transversely-extending port of the valve shaft when positioned within a valve well of a valve assembly.

Optionally, the cylindrical seal member defines one or more suction bleed passages axially along the cylindrical seal member between the valve shaft and the cylindrical seal member. Optionally, the valve shaft defines a bleed port therethrough in fluid communication with the one or more bleed passages.

Optionally, the valve shaft assembly further includes a shaft seal member on the valve shaft adjacent the distal end of the valve shaft.

In accordance with various principles of the present disclosure, a valve component for a medical valve assembly has a cylindrical seal member having an axial bore therethrough configured for extending circumferentially around a valve shaft and defining a transversely-extending suction-source communication port in fluid communication with a transversely-extending valve-shaft-suction-source port defined transversely through the valve shaft, with the valve-shaft-suction-source port in fluid communication with a valve-shaft-suction-application port extending axially through the valve shaft.

Optionally, the cylindrical seal member includes circumferential seal elements extending circumferentially around the cylindrical seal member and radially outwardly from the outer surface of the cylindrical seal member proximal and distal to the circumferential seal elements. Optionally, the circumferential seal elements form seals proximal and distal to the valve-shaft-suction-source port and the valve-well-suction-source port to create a sealed space extending circumferentially around the valve shaft to seal fluid communication through the valve assembly between a suction source fluidly in communication with the valve-well-suction-source port and a suction application device fluidly in communication with the valve-well-suction-application port.

Optionally, the cylindrical seal member defines one or more suction bleed passages axially along the cylindrical seal member and configured to be positioned between the cylindrical seal member and the valve shaft and in fluid communication with air outside the valve assembly. Optionally, the cylindrical seal member is configured to seal between the one or more bleed passages therethrough and a suction-source port defined in a valve well of the valve assembly.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to, e.g., fit within a working channel of a delivery catheter or endoscope. In the figures, identical or nearly identical or equivalent elements are typically represented by the same reference characters, and similar elements are typically designated with similar reference numbers, with redundant description omitted for the sake of brevity and convenience. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1 illustrates a perspective view of an example of an embodiment of an endoscope with one or more valves formed in accordance with aspects of the present disclosure.

FIG. 2 and FIG. 3 illustrate perspective views of an example of an embodiment of a valve assembly formed in accordance with various principles of the present disclosure, such as for an endoscope.

FIG. 4A illustrates a cross-sectional view, such as along line IV-IV of a valve assembly as illustrated in FIG. 3, of an example of an embodiment of a valve assembly in an off or closed configuration.

FIG. 4B illustrates a cross-sectional view similar to that of FIG. 4A, but with the valve assembly in an on or open configuration.

FIG. 5 illustrates a perspective view of an example of an embodiment of a valve shaft formed in accordance with various principles of the present disclosure for a valve assembly such as for an endoscope.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and/or closest to a delivery device, and “distal” refers to the direction or location furthest from the user, such as when using the device (e.g., introducing the device into a patient, or during implantation, positioning, or delivery), and/or closest to a delivery device. “Longitudinal” means extending along the longer or larger dimension of an element. A “longitudinal axis” extends along the longitudinal extent of an element, though is not necessarily straight and does not necessarily maintain a fixed configuration if the element flexes or bends, and “axial” generally refers to along the longitudinal axis. However, it will be appreciated that reference to axial or longitudinal movement with respect to the above-described systems or elements thereof need not be strictly limited to axial and/or longitudinal movements along a longitudinal axis or central axis of the referenced elements. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a channel, a cavity, or a bore. As used herein, a “channel” or “bore” or “passage” is not limited to a circular cross-section. As used herein, a “free end” of an element is a terminal end at which such element does not extend beyond. It will be appreciated that terms such as at or on or adjacent or along an end may be used interchangeably herein without intent to limit unless otherwise stated, and are intended to indicate a general relative spatial relation rather than a precisely limited location.

Various medical devices include valve assemblies to regulate or control fluid delivery (irrigation) or fluid suction (aspiration) with respect to an anatomical site. Although the present disclosure describes suction valves, it will be appreciated that the principles of the present disclosure need not be so limited.

A suction valve assembly of a medical device is arranged to apply suction from a suction source to an anatomical site via a flexible tubular element which is configured and positionable with respect to the anatomical site. The medical device may be an endoscope, and the flexible tubular element may be an insertion tube of the endoscope. The suction source may be a pump or other mechanism creating a vacuum to be applied to the anatomical site via the flexible tubular element. In the off configuration of the valve assembly, fluid communication between the suction source and the flexible tubular element is cut off or blocked so that suction is not applied to the anatomical site, and the valve may be considered to be in a closed configuration. In the on configuration of the valve assembly, the suction source is fluidly coupled with the flexible tubular element, such as to aspirate an anatomical site, and the valve may be considered to be in an open configuration.

Valve assemblies of medical devices may be mounted with respect to a control handle, and typically include an actuatable member movable with respect to the control handle to shift the valve assembly between the off configuration and the on configuration. The actuatable member may include a valve shaft and a user-engagement element which are movable with respect to a valve well formed in, or formed and positioned within, the control handle. Holes may be formed in the valve shaft to form channels which may be selectively aligned with ports in the valve well to selectively place the suction source in and out of fluid communication with the flexible tubular element of the device to apply suction to an anatomical site or to not apply suction. Various control handles have different arrangements of ports and flow paths placing the suction source in and out of fluid communication with the flexible tubular element to be directed to the anatomical site. Accordingly, various arrangements of holes within valve shafts may place ports to suction sources and ports to a flexible tubular element (directed to a patient) in and out of alignment with each other.

For the sake of convenience, and without intent to limit, reference is made herein to a valve assembly for a suction valve of an endoscope. The flexible tubular element of the endoscope is referenced herein as an insertion tube, and is generally positionable within a patient, such as within an organ, body lumen/passageway, cavity, etc. (reference being made herein to any or other such anatomical sites without intent to limit) The insertion tube defines one or more lumens therethrough configured for passage of materials, instruments, tools, devices, etc., through the working channel to an anatomical site. For instance, the lumens may include a suction lumen, an irrigation lumen, a working channel, and a visualization lumen (e.g., for a light guide, optic fiber, camera element, etc.). The present disclosure describes valve assemblies usable in a control handle with a suction-source port fluidly communicating with a laterally-extending suction channel to a suction source. More particularly, the laterally-extending suction channel extends transverse to the direction of actuation movement of the actuatable member of the valve assembly, and thus transverse to the longitudinal axis of the valve shaft. The suction channel and suction port are selectively moved in and out of fluid communication with a suction-application port fluidly communicating with an axially-extending suction-application port leading to the suction lumen through the insertion tube, and to an anatomical site when the endoscope is in use. More particularly, the axially-extending suction-application port is generally axially aligned with the direction of actuation movement of the actuatable member of the valve assembly and thus axially aligned with (optionally coaxial with) the longitudinal axis of the valve shaft. When the valve assembly is in an off configuration, the actuatable member is in a first position with the valve shaft blocking fluid communication to the suction-application port. When the valve assembly is in the on configuration, the valve shaft places the suction-application port in fluid communication with the suction-source port. Typically, the actuatable member of the valve assembly is biased into the off configuration, such as with a biasing element, so that suction is only applied when the medical professional intends to apply suction, such as by pressing on the actuatable member.

Seals are provided and are generally required to insure tight seals in the valve assembly. In particular, seals are provided to ensure that vacuum pressure does not affect the insertion tube when the distal end of the insertion tube is located in an anatomical site and suction is not required or desired. Moreover, seals are provided to prevent leakage of vacuum pressure which would affect the efficacy of suction applied to the anatomical site by the insertion tube. The present disclosure provides an improved valve assembly with improved sealing capability and optionally improved bleeding/venting capability as well. Whereas seal elements of prior art valves have been provided circumferentially around the ports to the flow channels (the holes in the valve shaft and/or the holes in the valve well leading to flow channels), the present disclosure provides seals circumferentially around the valve shaft. The prior art seals positioned circumferentially around ports transverse to the actuation axis along which a valve shaft moves may exhibit wear or may even become displaced by repeated movement of the valve shaft with respect to the valve well. More particularly, axial movement of the valve shaft with respect to the valve well results in lateral movement of the seal with respect to the flow path axis of the port about which the seal is positioned. In contrast, in accordance with various principles of the present disclosure, a valve seal is positioned circumferentially around a valve shaft as well as circumferentially around the suction portions, and optionally also is secured in place against axial shifting with respect to the valve shaft, improving sealing achieved by the valve assembly. In some embodiments, the valve seal is a cylindrical seal extending along the outer circumference of the valve shaft with a hole for fluidly communicating the suction-source port in the valve well with and the suction-source port in the valve shaft. In some embodiments, the valve seal includes venting/bleed passages to allow bleeding of vacuum pressure when suction is not being applied to an anatomical site yet the suction source is continuously being run during a procedure. The valve shaft with the valve seal may be considered a valve shaft assembly.

Various features and embodiments of valve devices (including, without limitation, components and assemblies), systems, and methods will now be described with reference to examples illustrated in the accompanying drawings. Reference in this specification to “one embodiment,” “an embodiment,” “some embodiments”, “other embodiments”, etc. indicates that one or more particular features, structures, concepts, and/or characteristics in accordance with principles of the present disclosure may be included in connection with the embodiment. However, such references do not necessarily mean that all embodiments include the particular features, structures, concepts, and/or characteristics, or that an embodiment includes all features, structures, concepts, and/or characteristics. Some embodiments may include one or more such features, structures, concepts, and/or characteristics, in various combinations thereof. It should be understood that one or more of the features, structures, concepts, and/or characteristics described with reference to one embodiment can be combined with one or more of the features, structures, concepts, and/or characteristics of any of the other embodiments provided herein. That is, any of the features, structures, concepts, and/or characteristics described herein can be mixed and matched to create hybrid embodiments, and such hybrid embodiment are within the scope of the present disclosure. Moreover, references to “one embodiment,” “an embodiment,” “some embodiments”, “other embodiments”, etc. in various places in the specification are not necessarily all referring to the same embodiment, nor are separate or alternative embodiments necessarily mutually exclusive of other embodiments. It should further be understood that various features, structures, concepts, and/or characteristics of disclosed embodiments are independent of and separate from one another, and may be used or present individually or in various combinations with one another to create alternative embodiments which are considered part of the present disclosure. Therefore, the present disclosure is not limited to only the embodiments specifically described herein, as it would be too cumbersome to describe all of the numerous possible combinations and subcombinations of features, structures, concepts, and/or characteristics, and the examples of embodiments disclosed herein are not intended as limiting the broader aspects of the present disclosure. It should be appreciated that various dimensions provided herein are examples and one of ordinary skill in the art can readily determine the standard deviations and appropriate ranges of acceptable variations therefrom which are covered by the present disclosure and any claims associated therewith. The following description is of illustrative examples of embodiments only, and is not intended as limiting the broader aspects of the present disclosure.

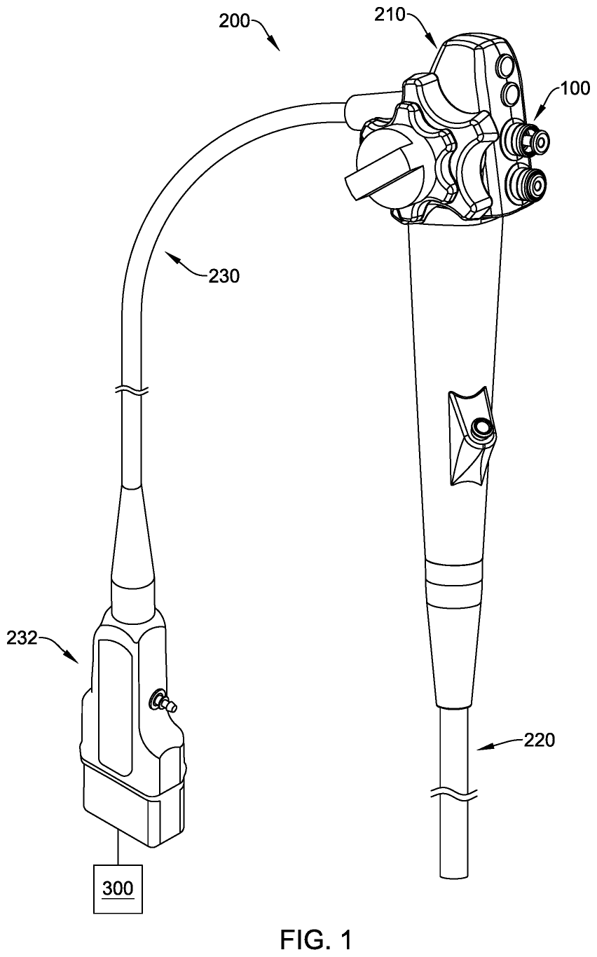

Turning now to the drawings, an example of an embodiment of a valve assembly 100 formed in accordance with various principles of the present disclosure is illustrated in FIG. 1 as provided in an example of an embodiment of an endoscope 200. It will be appreciated that the endoscope 200 is an example of an embodiment in which principles of the present disclosure may be applied, and that various principles of the present disclosure are applicable to other medical instruments to control fluid flow with respect thereto, the details of which are not critical to the present disclosure.

The valve assembly 100 is mounted with respect to a control handle 210 of the endoscope 200 to regulate the flow of materials (e.g., fluid) between the insertion tube 220 of the endoscope 200 and a suction source 300. The endoscope 200 has a connector cord 230 extending to a scope connector 232 with which the endoscope 200 (and valve assembly 100) may be fluidly coupled with the suction source 300. The connector cord 230 may be alternatively referenced herein as an umbilical cord, umbilicus, universal cord, etc., without intent to limit The scope connector 232 may also couple the endoscope 200, via the connector cord 230, with a variety of components, devices, etc., such as a fluid source (to supply air, carbon dioxide, water, saline, or other gases or liquids), electrical connections, light sources, visualization elements (e.g., optic fibers, cameras, etc.), or other components, devices, etc., usable with the endoscope 200. The insertion tube 220 has a fluid lumen extending therethrough to a distal end which is positionable (insertable, navigable, etc.) with respect to an anatomical site (e.g., within a patient). Similarly, the connector cord 230 has a fluid lumen extending therethrough to fluidly couple the suction source 300 (e.g., via the scope connector 232) with the control handle 210. The fluid lumens through the insertion tube 220 and the connector cord 230, and distal end of the insertion tube 220, may be well-known features formed in a manner known to those of ordinary skill in the art and are not illustrated to simplify the drawings by eliminating details in the illustration of the endoscope 200 in FIG. 1 which are not necessary for understanding the present disclosure.

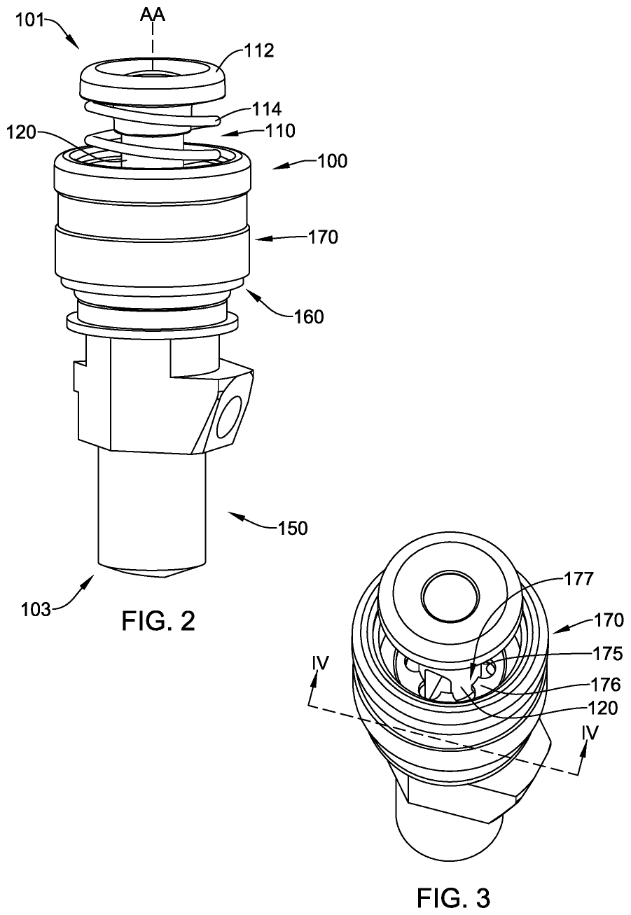

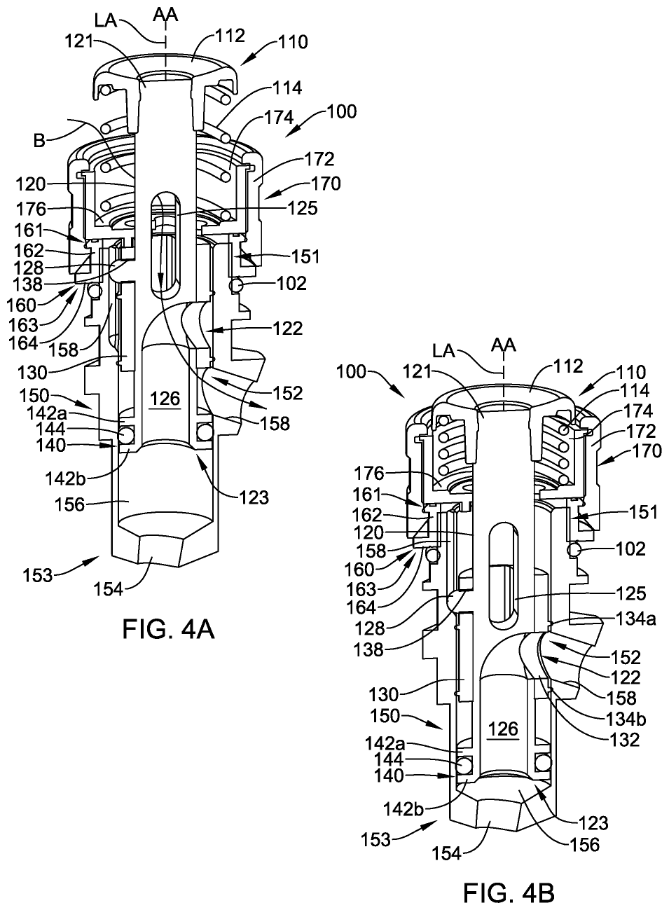

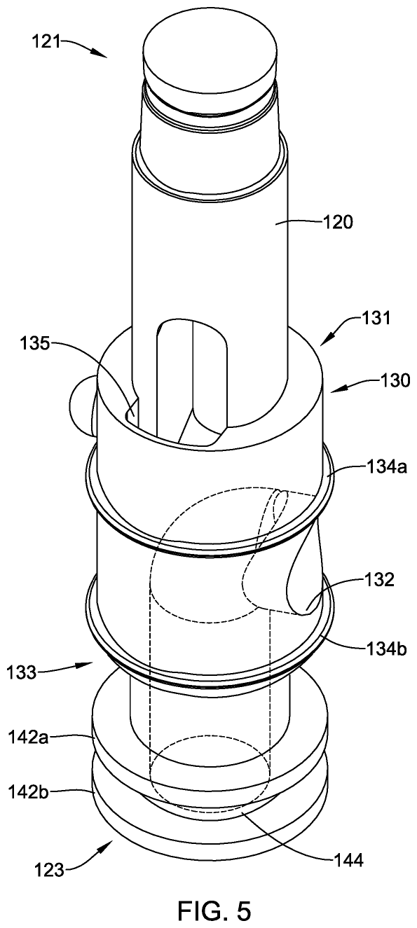

An example of an embodiment of a valve assembly 100 formed in accordance with various principles of the present disclosure is illustrated in FIG. 2, FIG. 3, FIG. 4A, and FIG. 4B, in isolation from an endoscope (such as the endoscope 200 illustrated in FIG. 1). In the illustrated example of an embodiment, the valve assembly 100 includes an actuatable member 110 movable with respect to a valve well 150 of the valve assembly 100. More particularly, the actuatable member 110 includes a valve shaft 120 movable within and with respect to the valve well 150, with a portion of the valve shaft 120 movable within the valve well 150. Seal members 130 and 140 are provided on the valve shaft 120 to seal the suction paths through the valve assembly 100 in the on and off configurations of the valve assembly 100, as described in further detail below.

The illustrated example of an embodiment of a valve well 150 is formed separately from and inserted into the control handle 210 of an endoscope 200 such as illustrated in FIG. 1, although the present disclosure is not limited in this regard. A valve well nut 160, illustrated in FIG. 2 and in further detail in the cross-sectional views of FIG. 4A and FIG. 4B, may hold the valve well 150 in place with respect to a control handle (such as the control handle 210 illustrated in FIG. 1), such as in a manner known to those of ordinary skill in the art. A seal 162, such as illustrated in FIG. 4A and FIG. 4B, may be provided between the valve well nut 160 and the proximal end 151 of the valve well 150, such as to create a seal between the valve well nut 160 and the control handle (and the valve well 150 therein). An example of an embodiment of a cap 170 is illustrated as coupled with respect to the valve well 150 via the valve well nut 160, however the present disclosure is not limited to the illustrated arrangement. The cap 170 may provide various features for assembly and use of the valve assembly 100, such as guides for actuation movement of the actuatable member 110, orientation features for the actuatable member 110, suction bleed passages through the valve assembly 100, and other features, as described in further detail below. In some embodiments, the cap 170 is a single piece element, while in some embodiments, the cap 170 is a two-piece element with a skirt or sleeve 172 having a cup 174 inserted therein.

The actuatable member 110 of the valve assembly 100 includes a user-engagement element 112 configured to facilitate engagement and actuation of the actuatable member 110 at a proximal end 101 of the valve assembly 100. In the example of an embodiment illustrated in FIG. 2, FIG. 3, FIG. 4A, and FIG. 4B, the user-engagement element 112 is a button coupled to a proximal end 121 of the valve shaft 120 (e.g., in any of a variety of manners known to those of ordinary skill in the art), although the user-engagement element 112 may, instead, be integrally formed with the valve shaft 120. In some embodiments, a separately-formed user-engagement element 112 allows the user-engagement element 112 to be formed of a material comfortable to be pressed by a user's finger, such as a compliant elastomeric material. The actuatable member 110 is movable along an actuation axis AA, as may appreciated with reference to FIG. 4A and FIG. 4B. A biasing element 114 may be provided to maintain the actuatable member 110 in a neutral position. The biasing element 114 may be a coil spring or other element capable of holding elements apart yet allowing such elements to be selectively moved together upon application of force to one of the elements and/or to the biasing element. The biasing element 114 may be positioned between an underside of the user-engagement element 112 and a base 176 of the cap 170 (either a radially extending section of the skirt 172 or of the cup 174 within the skirt 172), such as in a manner known by those of ordinary skill in the art. It will be appreciated that the biasing element 114 is not illustrated in the perspective view of FIG. 3 to facilitate illustration of features of the cap 170 described below. The actuatable member 110 may be biased by the biasing element 114 into a neutral position maintaining the valve assembly 100 in one of an on or off configuration (typically an off configuration). In the illustrated examples of embodiments, the actuatable member 110 of the valve assembly 100 is shifted from a neutral position, in which the valve assembly 100 is in an off configuration, to shift the valve assembly 100 into on configuration. For instance, the user-engagement element 112 may be shifted from a position proximal to the proximal end 101 of the valve assembly 100 distally toward the distal end 103 of the valve assembly 100. However, principles of the present disclosure may be applied to other arrangements as well.

When the actuatable member 110 is in an off position, the valve assembly 100 is in an off configuration, and suction from a suction source fluidly coupled with the valve assembly 100 is not supplied by the valve assembly 100. When the actuatable member 110 is shifted to an on position, the valve assembly 100 is shifted to an on configuration and suction from the suction source is supplied by the valve assembly 100, such as to a suction application device fluidly coupled therewith. In the context of an endoscope 200 as illustrated in FIG. 1, the suction application device is the insertion tube 220, although a valve assembly 100 formed in accordance with various principles of the present disclosure may be coupled with other types of suction application devices known to those of ordinary skill in the art. The example of an embodiment of a valve assembly 100 illustrated in FIG. 4A and FIG. 4B has a valve well 150 with a valve-well-suction-source port 152 in fluid communication with a suction source (such as a suction source 300 as illustrated in FIG. 1), and a valve-well-suction-application port 154 in fluid communication with a suction application device (such as an insertion tube 220 as illustrated in FIG. 1). In the illustrated example of an embodiment, the valve-well-suction-source port 152 extends transversely with respect to the actuation axis A, and the valve-well-suction-application port 154 extends generally along the actuation axis A. Furthermore, in the example of an embodiment of a valve assembly 100 illustrated in FIG. 4A and FIG. 4B, the valve shaft 120 of the valve assembly 100 has a valve-shaft-suction-source port 122 movable into and out of alignment with the valve-well-suction-source port 152 (and thus also extending transversely with respect to the actuation axis A) to place the suction source in fluid communication with the suction application device via the valve assembly 100. The valve shaft 120 also includes a valve-shaft-suction-application port 124 in fluid communication with a valve-well-suction-application port 154 (and thus also extending generally along the actuation axis A) via which the valve assembly 100 may apply suction. When the actuatable member 110 is in a neutral position, the valve shaft 120 is in an off position and the valve assembly 100 is in an off configuration with the valve-shaft-suction-source port 122 out of alignment with the valve-well-suction-source port 152. Accordingly, the suction source is not in fluid communication with the valve-shaft-suction-application port 124 or the valve-well-suction-application port 154, and the valve assembly 100 does not apply suction. When the actuatable member 110 is actuated to move the valve assembly 100 into an on configuration, the valve shaft 120 is shifted into an on position to align the valve-shaft-suction-source port 122 with the valve-well-suction-source port 152 to place the suction source in fluid communication with the valve shaft suction channel 126 extending generally axially through the valve shaft 120 and thus in fluid communication with the valve-shaft-suction-application port 124 and the valve-well-suction-application port 154.

In the example of an embodiment of a valve assembly 100 illustrated in FIG. 4A and FIG. 4B, the suction source is coupled to the valve assembly 100 laterally with respect to the actuation axis AA of the actuatable member 110, and suction is applied generally axially with respect to the actuation axis AA. As such, the valve-shaft-suction-source port 122 is a side port defined in a side wall of the valve shaft 120, and the valve-shaft-suction-application port 124 is an axial port defined through the distal end 123 of the valve shaft 120 Likewise, the valve-well-suction-source port 152 of the valve assembly 100 is a side port fluidly coupled with the suction source laterally with respect to the actuation axis AA of the actuatable member 110 (and generally axially with respect to the valve-shaft-suction-source port 122), and the valve-well-suction-application port 154 is an axial port applying suction from the valve assembly 100 in a generally axial direction along the actuation axis AA (and generally axially with respect to the valve-shaft-suction-application port 124). The longitudinal axis LA of the valve shaft 120 generally extends along (parallel to or coaxial with) the actuation axis AA of the actuatable member 110, and reference herein may be made alternately to the longitudinal axis LA of the valve shaft 120 or the actuation axis AA of the actuatable member 110 without intent to limit

When the valve-shaft-suction-source port 122 is aligned with the valve-well-suction-source port 152, suction flows from the suction source through the suction-source ports 122, 152 in a direction generally transverse to the longitudinal axis of the valve shaft 120, and is then directed axially to apply suction via the valve-shaft-suction-application port 124 and the valve-well-suction-application port 154. As such, the suction channel 126 extending through the valve shaft 120, between the valve-shaft-suction-source port 122 and the valve-shaft-suction-source port 122, is generally elbow-shaped with a bend therealong to transition suction applied transversely into the valve shaft 120 (via the valve-shaft-suction-source port 122) to suction applied axially from the valve shaft 120 (via the valve-shaft-suction-application port 124).

In accordance with various principles of the present disclosure, the valve assembly 100 has improved seal members and an improved sealing arrangement, such as illustrated in FIG. 4A and FIG. 4B. The illustrated seal arrangement includes a cylindrical seal member 130 arranged on the valve shaft 120 with respect to the valve-shaft-suction-source port 122, and a shaft seal member 140 arranged on the valve shaft 120 with respect to the valve-shaft-suction-application port 124. In accordance with various principles of the present disclosure, the cylindrical seal member 130 is formed separately from the valve shaft 120 and positioned circumferentially around a portion of valve shaft 120. The cylindrical seal member 130 is formed of a sealing material capable of forming a seal between the valve shaft 120 and the valve well 150 (e.g., a thermoplastic elastomer or silicone). Typically, the cylindrical seal member 130 is relatively flexible, and the valve shaft 120 is formed of a more rigid material (e.g., a more rigid plastic, or a metal) to resist deformation which may result from repeated use, continuous axial movement, force applied thereto by the biasing element 114, etc. The example of an embodiment of a shaft seal member 140 illustrated in FIG. 4A, FIG. 4B, and FIG. 5 is formed with a proximal flange 142a spaced apart from a distal flange 142b defining a space therebetween for a seal member 144. The flanges 142a, 142b may be formed from a rigid material (e.g., integrally with the valve shaft 120) and the seal member 144 may be formed of an elastomeric sealing material (e.g., o-ring). However, other configurations are within the scope and spirit of the present disclosure.

The configuration and arrangement of the cylindrical seal member 130 and the shaft seal member 140 provide improved sealing of suction flow within the valve assembly 100 when the valve assembly 100 is in the on configuration. In some instances, the suction source fluidly coupled with the valve assembly 100 continuously applies suction to the valve assembly 100 (i.e., is continuously on). In such case, the actuatable member 110 of the valve assembly 100 does not actuate the suction source, but simply places the suction source into and out of fluid communication with the suction application portion of the valve assembly 100, and, typically, a suction application device fluidly coupled with the suction application portion of the valve assembly 100. For example, in the context of the endoscope 200 illustrated in FIG. 1, operation of the actuatable member 110 of the valve assembly 100 selectively applies or cuts off suction with respect to an anatomical site with respect to which the insertion tube 220 is positioned. Accordingly, the improved seal arrangement within the valve assembly 100 may not simply maintain suction through the valve assembly 100 when the valve assembly 100 is in an on configuration, but may also improve isolation of suction to seal the suction source from the suction application device when the valve assembly 100 is in the off configuration.

When the valve assembly 100 is in an off configuration, the valve-shaft-suction-source port 122 is axially out of alignment with the valve-well-suction-source port 152, as illustrated in FIG. 4A. The cylindrical seal member 130 is positioned radially between the valve shaft 120 and the valve well 150, and axially between the valve-shaft-suction-source port 122 and the valve-well-suction-source port 152 to seal the valve-shaft-suction-source port 122 from suction applied from the suction source to the valve-well-suction-source port 152. Furthermore, by sealing the valve-shaft-suction-source port 122, the cylindrical seal member 130 seals the valve shaft suction channel 126 with respect to the suction source, and thereby seals the valve-shaft-suction-application port 124 and the valve-well-suction-application port 154 from fluid communication with the suction source. Thus, although in the illustrated example of an embodiment of a valve assembly 100, the valve-shaft-suction-source port 122 remains in fluid communication with the valve-well-suction-application port 154, via the suction channel 126 extending through the valve shaft 120, no vacuum pressure reaches the valve-shaft-suction-application port 124 to apply suction to the anatomical site in the patient via the valve-well-suction-application port 154. Additionally, the shaft seal member 140 is positioned radially between the valve shaft 120 and the valve well 150, and axially between the valve-well-suction-source port 152 and the valve-well-suction-application port 154 to seal the valve-well-suction-application port 154 from suction from the valve-well-suction-source port 152. Accordingly, the suction source is not in fluid communication with the valve-well-suction-application port 154 and the valve assembly 100 does not apply suction (such as to an insertion tube 220 of an endoscope 200 and/or to an anatomical site).

It is generally desirable to limit suction applied by the valve assembly 100 to instances

when suction is desired, and to limit, and preferably eliminate, suction force when suction is not desired. For instance, in certain endoscopic procedures, it is desirable to maintain an anatomical site insufflated to improve visualization of the target site of the procedure, and/or to irrigate a target site, such as supplying fluid to the target site. Suction may be limited to reducing the supplied fluid in certain instances, and/or to remove other materials (e.g., biological materials) from the target site. A cylindrical seal member 130 formed in accordance with various principles of the present disclosure provides various benefits over prior seals of valve assemblies, such as with similarly configured valve port and flow channel arrangements, as may be appreciated with reference to the example of an embodiment of a cylindrical seal member 130 illustrated in cross-section FIG. 4A and FIG. 4B, and in a perspective view in FIG. 5.

For instance, as may be appreciated with reference to FIG. 5, the cylindrical seal member 130 is provided circumferentially around and extending axially along the valve shaft 120. A suction-source communication port 132 is defined through the cylindrical side wall for fluid access through the cylindrical seal member 130 to the valve-shaft-suction-source port 122. Such configuration forms an extended seal circumferentially and axially around the valve-shaft-suction-source port 122 defined in the valve shaft 120, in contrast with the limited sealing extent typically provided by prior art o-rings provided around the perimeter of a valve- shaft-suction-source port 122. As may be appreciated with reference to FIG. 5, a generally cylindrical seal member 130, extending circumferentially around the valve shaft 120 as well as axially along the valve shaft 120 beyond the immediate vicinity of the valve-shaft-suction-source port 122, provides greater stability with respect to the cylindrical seal member 130 as the valve shaft 120 is actuated up and down along the actuation axis AA within and with respect to the valve well 150. Because of the increased dimensions of the cylindrical seal member 130 relative to prior art seal members (such as o-rings positioned around the valve-shaft-suction-source port 122 and/or the valve-well-suction-source port 152), the cylindrical seal member 130 is better able to maintain its position on the valve shaft 120 and its sealing position with respect to the valve-shaft-suction-source port 122 and/or the valve-well-suction-source port 152 without deforming or suffering from the type of wear exhibited by prior art seals. Moreover, because the cylindrical seal member 130 extends cylindrically around the valve shaft 120, the cylindrical seal member 130 provides circumferential sealing between the valve-well-suction-source port 152 and the valve-shaft-suction-source port 122 when the valve assembly 100 is in the off configuration.

As may be appreciated, the exterior surface of the cylindrical seal member 130 contacts the interior surface of the valve-well channel 156 within the valve well 150 to form a seal between the valve shaft 120 and the valve well 150. In some embodiments, the seal established by the cylindrical seal member 130 with respect to the valve-shaft-suction-source port 122 is provided by axially-spaced-apart circumferential seal elements 134a, 134b extending circumferentially around and radially-outwardly from the surrounding outer surface of the cylindrical seal member 130 extending above and below each of the circumferential seal elements 134a, 134b. As may be appreciated, the radially-outward surfaces of the circumferential seal elements 134a, 134b contact the inner surface of the valve well 150, thereby reducing the overall surface to surface contact area between the cylindrical seal member 130 and the interior of the valve well 150. Such configuration may reduce overall friction generated as the actuatable member 110 moves between the off and on positions to actuate the valve assembly 100 (which may occur multiple times during use of the valve assembly 100), thereby reducing friction between, reducing risk of deformation, wear, displacement, etc., exhibited by prior art seals.

As described above, in some instances, the suction source is continuously running/on during use of a valve assembly 100 formed in accordance with various principles of the present disclosure, even if suction is not being applied by the valve assembly 100 (e.g., when the valve-well-suction-source port 152 is out of alignment with the valve-shaft-suction-source port 122). In such instances, it may be desirable to vent or bleed vacuum pressure generated within the valve assembly 100 from the suction source (via the valve-well-suction-source port 152) when the valve assembly 100 is in the off configuration. Provision of a cylindrical seal member 130 separately formed from the valve shaft 120 facilitates sealing of the valve-shaft-suction-source port 122 with respect to the valve-well-suction-source port 152 while also allowing venting or bleeding of suction via the cylindrical seal member 130 when the valve assembly 100 is in the off configuration. In accordance with various principles of the present disclosure, a cylindrical seal member 130 has an outer diameter larger than the outer diameter of the valve shaft 120, as may be appreciated with reference to FIG. 4A, FIG. 4B, and FIG. 5. One or more suction bleed passages 135 may be defined axially along the cylindrical seal member 130 between the exterior of the valve shaft 120 and the interior of the cylindrical seal member 130 and radially-outwardly of the bore 137 defined axially through the cylindrical seal member 130 and through which the valve shaft 120 extends. Ambient air may enter the seal suction bleed passages 135 via the proximal end 131 of the cylindrical seal member 130, and travel axially along the seal suction bleed passages 135 to exit along the distal end 133 of the cylindrical seal member 130 to bleed the valve-well-suction-source port 152 therealong when the valve shaft 120 is in the off position illustrated in FIG. 4A. As may further be appreciated with reference to FIG. 4A, the path B for bleeding ambient air to the suction source may extend through a bleed port 125 extending through the valve shaft 120. The bleed port 125 of the valve shaft 120 may extend above the base 176 of the cap 170 (at least when the valve shaft 120 is in the off position illustrated in FIG. 4A) to allow the bleed path B to extend from outside the valve assembly 100 (above the cap 170), through the valve shaft 120, and into the valve assembly 100, below the cap 170. The valve shaft bleed port 125 need not extend above the cap 170 when the valve shaft 120 is in the on position, as illustrated in FIG. 4B, since bleeding of the suction source is not necessary when the valve assembly 100 is in the on configuration. Although not necessary if a bleed port 125 is provided through the valve shaft 120, the base 176 of the cap 170 optionally includes vent holes 175 providing a bleed path B therethrough. In some embodiments, flats may be formed axially along the valve shaft 120 to define the bleed path B therealong from the proximal end 121 of the valve shaft 120 to the distal end 123 of the valve shaft 120. As may be appreciated, the outer surface of the distal end 133 of the cylindrical seal member 130 (and the circumferential seal elements 134a, 134b, if provided) seals the valve-shaft-suction-source port 122 from the ambient bleed path B when the valve shaft 120 is in the on position fluidly communicating the valve-shaft-suction-source port 122 with the valve-well-suction-source port 152, as illustrated in FIG. 4B. Moreover, the shaft seal member 140 seals the valve-well-suction-application port 154 from the bleed path B, which prevents loss of suction when the valve assembly 100 is in the on configuration.

In the on configuration of the valve assembly 100, such as illustrated in FIG. 4B, the valve shaft 120 is positioned with respect to the valve well 150 to axially align and place the valve-shaft-suction-source port 122 in fluid communication with the valve-well-suction-source port 152. Since the valve-shaft-suction-application port 124 remains aligned and in fluid communication with the valve-well-suction-application port 154, and remains in fluid communication with the valve-shaft-suction-source port 122 via the valve shaft suction channel 126, once the valve-shaft-suction-source port 122 is aligned into fluid communication with the valve-well-suction-source port 152, suction may be applied by the valve assembly 100 via the valve-well-suction-application port 154, such as to an anatomical site. The seal members 130, 140 seal the suction flow path in the on configuration of the valve assembly 100 by sealing the fluid communication of the valve-shaft-suction-application port 124 and the valve-well-suction-source port 152 with respect to each other as well as with respect to the valve shaft suction channel 126. In particular, the port 132 of the cylindrical seal member 130 seals around the valve-shaft-suction-source port 122 and the valve-well-suction-source port 152 to seal the suction path therethrough. The shaft seal member 140 continues to provide a seal with respect to the valve-well-suction-application port 154 to limit fluid communication therewith to fluid communication with the valve shaft suction channel 126 and the valve-shaft-suction-application port 124, and to seal the valve-well-suction-application port 154 from fluid communication with the bleed path B through the cylindrical seal member 130. As such, suction applied by the valve assembly 100 is not diminished by the bleed path B within the valve assembly 100.

In some embodiments, the seal established by the cylindrical seal member 130 with respect to the valve-shaft-suction-source port 122 and/or the valve-well-suction-source port 152 when the valve assembly 100 is in the on configuration is provided between circumferential seal elements 134a, 134b extending circumferentially around and radially outwardly from the cylindrical seal member 130 and the valve shaft 120, such as described above. The proximal seal element 134a creates a proximal seal (closer to the proximal end 101 of the valve assembly 100) with respect to the valve-well-suction-source port 152, and the distal seal element 134b creates a distal seal (closer to the distal end 103 of the valve assembly 100) with respect to the valve-well-suction-source port 152. The circumferential seal elements 134a, 134b thereby define a sealed enclosed space extending circumferentially around the valve shaft 120, radially between the valve shaft 120 and the valve well 150, and axially between the circumferential seal elements 134a, 134b. The valve region created by the circumferential seal elements 134a, 134b extending circumferentially around the valve shaft 120 provides a larger sealing area around the valve-shaft-suction-source port 122 and the valve-well-suction-source port 152 than provided by seals extending circumferentially around such ports as in prior art valve assemblies. Sufficient space can be provided between the circumferential seal elements 134a, 134b such that even if the circumferential seal elements 134a, 134b shift a bit or if the valve-shaft-suction-source port 122 and the valve-well-suction-source port 152 become slightly axially out of alignment, the seal is maintained.

It will be appreciated that alignment of the valve-shaft-suction-source port 122 and valve-well-suction-source port 152 is desirable for effective application of vacuum to an anatomical site by the valve assembly 100. In some embodiments, the valve shaft 120 and the cap 170 having alignment features, such as mating shapes (e.g., non-circular shapes) which orient the valve shaft 120 with respect to valve well 150 to maintain alignment between the laterally-opening valve-shaft-suction-source port 122 and the valve-well-suction-source port 152, such as in manners known to those of ordinary skill in the art. Additionally or alternatively, the valve shaft 120 may include an alignment member 128 extending radially into an axially-extending alignment slot 158 in the valve well 150, such as illustrated in FIG. 4A and FIG. 4B. Engagement of the alignment member 128 with the alignment slot 158 allows axial/longitudinal shifting of the actuatable member 110 between on and off positions to shift the valve assembly 100 between on and off configurations, while maintaining rotational alignment of the valve-shaft-suction-source port 122 and the valve-well-suction-source port 152 when the valve shaft 120 is in the on configuration. In particular, the alignment member 128 rotationally fixes the valve shaft 120 with respect to the valve well 150 to maintain the rotational alignment of the valve-shaft-suction-source port 122 with respect to the valve-well-suction-source port 152 (so that the valve shaft 120 does not rotate the valve-shaft-suction-source port 122 out of alignment with the valve-well-suction-source port 152 when the valve shaft 120 is in the on position). Moreover, the alignment slot 158 may provide an upper limit stop to the axial travel of the valve shaft 120, such as in the off position illustrated in FIG. 4A (e.g., holding the valve shaft 120 within the valve well 150), and a lower limit stop to the valve shaft 120, such as in the on position illustrated in FIG. 4B (at which position the valve-shaft-suction-source port 122 is aligned with the valve-well-suction-source port 152). It will be appreciated that the solid height of the biasing element 114 (when the biasing element 114 bottoms out) may also determine the distal distance the actuatable member 110 may be shifted distally with respect to the distal end 103 of the valve assembly 100, and thus may contribute to ensuring alignment of the valve-shaft-suction-source port 122 and the valve-well-suction-source port 152.

As may be further appreciated with reference to FIG. 4A and FIG. 4B, the alignment member 128 extends through a hole 138 in the cylindrical seal member 130 to extend from the valve shaft 120 into the alignment slot 158. As such, the alignment member 128 engages the cylindrical seal member 130 to maintain rotational and axial alignment of the cylindrical seal member 130 with respect to the valve shaft 120. Consequently, alignment of the suction-source communication port 132 through the cylindrical seal member 130 with respect to the valve-shaft-suction-source port 122 and the valve-well-suction-source port 152 is maintained as well. The alignment member 128 may secure a separately formed cylindrical seal member 130 with respect to the valve shaft 120 even if the cylindrical seal member 130 is not molded thereon.

Various further benefits of the various aspects, features, components, and structures of a valve shaft and associated seal members, as well as valve assemblies and endoscopes such as described above, in addition to those discussed above, may be appreciated by those of ordinary skill in the art.

It is to be understood by one of ordinary skill in the art that the present discussion is a description of illustrative examples of embodiments only, and is not intended as limiting the broader aspects of the present disclosure. It will be appreciated that principles of the present disclosure may be applied to various medical devices, instruments, tools, etc., such, without limitation, a variety of medical devices, instruments, tools, etc., for accessing anatomical sites and applying suction and/or irrigation thereto, including, for example, endoscopes, gastroscopes, duodenoscopes, catheters, ureteroscopes, bronchoscopes, colonoscopes, arthroscopes, cystoscopes, hysteroscopes, and the like, having integrated features for suction and/or irrigation of anatomical sites. Moreover, principles of the present disclosure may be applied to reusable or single-use devices, instruments, tools, etc.

All apparatuses and methods discussed herein are examples of apparatuses and/or methods implemented in accordance with one or more principles of this disclosure. These examples are not the only way to implement these principles but are merely examples, not intended as limiting the broader aspects of the present disclosure. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. For instance, various elements and components of a valve assembly described herein may be coupled or engaged directly or indirectly with one another, regardless of how such connections are depicted in the drawings. It should be apparent to those of ordinary skill in the art that variations can be applied to the disclosed devices, systems, and/or methods, and/or to the sequence of steps of the method described herein without departing from the concept, spirit, and scope of the disclosure. It will be appreciated that various features described with respect to one embodiment typically may be applied to another embodiment, whether or not explicitly indicated. The various features hereinafter described may be used singly or in any combination thereof. Therefore, the present invention is not limited to only the embodiments specifically described herein, and all substitutes and modifications apparent to those skilled in the art are deemed to be within the spirit, scope, and concept of the disclosure as defined by the appended claims.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and/or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as 10 used herein, refers to one or more of that entity. As such, the terms “a” (or “an”), “one or more” and “at least one” can be used interchangeably herein. As used in this specification and the appended claims, the term “or” is generally employed in its sense including “and/or” unless the content clearly dictates otherwise. As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, engaged, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the terms “comprises”, “comprising”, “includes”, and “including” do not exclude the presence of other elements, components, features, groups, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. A valve component for a medical valve assembly, said valve component comprising:
a valve shaft extendable through a valve well of a valve assembly and along an actuation axis for actuation movement along the actuation axis, and defining a valve-shaft-suction-source port extending transverse to the actuation axis, and a valve-shaft-suction-application port extending axially along the actuation axis and in fluid communication with the valve-shaft-suction-source port; and
a cylindrical seal member extending circumferentially around said valve shaft and defining a suction-source communication port in fluid communication with the valve-shaft-suction- source port;
wherein said valve shaft is axially movable within a valve well between an off position in which the valve assembly is in an off configuration with the valve-shaft-suction-source port not in fluid communication with the valve-well-suction-source port, and an on position in which the valve assembly is in an on configuration with the valve-shaft-suction-source port in fluid communication with the valve-well-suction-source port.

2. The valve component of claim 1, wherein said cylindrical seal member includes circumferential seal elements extending circumferentially around said cylindrical seal member and radially outwardly from the outer surface of said cylindrical seal member proximal and distal to said circumferential seal elements.

3. The valve component of claim 2, wherein said circumferential seal elements form seals proximal and distal to the valve-shaft-suction-source port and the valve-well-suction-source port to create a sealed space extending circumferentially around said valve shaft to seal fluid communication through the valve assembly between a suction source fluidly in communication with the valve-well-suction-source port and a suction application device fluidly in communication with the valve-well-suction-application port.

4. The valve component of claim 1, wherein said valve shaft has a proximal end extending away from the valve well for actuation by a user, and a distal end extending within the valve well, said valve assembly further comprising a shaft seal member adjacent the distal end of said valve shaft.

5. The valve component of claim 4, wherein said shaft seal member seals between the valve-well-suction-source port and the valve-well-suction-application port.

6. The valve component of claim 1, wherein said cylindrical seal member defines one or more suction bleed passages axially along said cylindrical seal member between said valve shaft and said cylindrical seal member.

7. The valve component of claim 6, wherein said one or more suction bleed passages are in fluid communication with air outside the valve assembly to allow air from outside the valve assembly to bleed a suction source in fluid communication with the valve-well-suction-source port when the suction source is running and the valve assembly is in the off configuration.

8. The valve component of claim 7, wherein said valve shaft defines a bleed port therethrough in fluid communication with air outside the valve assembly and with the one or more bleed passages.

9. The valve component of claim 8, wherein the one or more bleed passages extend axially through said cylindrical seal member, and said cylindrical seal member seals between the one or more bleed passages therethrough and the valve-well-suction-source port.

10. A valve shaft assembly for a valve assembly of a medical device, said valve shaft assembly comprising:
a valve shaft having a proximal end configured for engagement by a user, a distal end, and a longitudinal axis extending therebetween, said valve shaft defining a transversely-extending port extending transverse to the longitudinal axis, and an axially-extending port extending axially along longitudinal actuation axis, with the transversely-extending port in fluid communication with the axially-extending port; and
a flexible cylindrical seal member formed from a sealing material more flexible than said valve shaft, and extending circumferentially around said valve shaft and defining a transversely-extending port in fluid communication with the transversely-extending port in said valve shaft.

11. The valve shaft assembly of claim 10, wherein said cylindrical seal member includes circumferential seal elements extending circumferentially around said cylindrical seal member and radially outwardly from the outer surface of said cylindrical seal member proximal and distal to said circumferential seal elements.

12. The valve assembly of claim 11, wherein said circumferential seal elements are configured to define seals proximal and distal to the transversely-extending port of said valve shaft when positioned within a valve well of a valve assembly.

13. The valve shaft assembly of claim 10, wherein said cylindrical seal member defines one or more suction bleed passages axially along said cylindrical seal member between said valve shaft and said cylindrical seal member.

14. The valve shaft assembly of claim 13, wherein said valve shaft defines a bleed port therethrough in fluid communication with the one or more bleed passages.

15. The valve shaft assembly of claim 10, further comprising a shaft seal member on said valve shaft adjacent the distal end of said valve shaft.

16. A valve component for a medical valve assembly, said valve component comprising a cylindrical seal member having an axial bore therethrough configured for extending circumferentially around a valve shaft and defining a transversely-extending suction-source communication port in fluid communication with a transversely-extending valve-shaft-suction-source port defined transversely through the valve shaft, with the valve-shaft-suction-source port in fluid communication with a valve-shaft-suction-application port extending axially through the valve shaft.

17. The valve component of claim 16, wherein said cylindrical seal member includes circumferential seal elements extending circumferentially around said cylindrical seal member and radially outwardly from the outer surface of said cylindrical seal member proximal and distal to said circumferential seal elements.

18. The valve component of claim 17, wherein said circumferential seal elements form seals proximal and distal to the valve-shaft-suction-source port and the valve-well-suction-source port to create a sealed space extending circumferentially around said valve shaft to seal fluid communication through said valve assembly between a suction source fluidly in communication with the valve-well-suction-source port and a suction application device fluidly in communication with the valve-well-suction-application port.

19. The valve component of claim 16, wherein said cylindrical seal member defines one or more suction bleed passages axially along said cylindrical seal member and configured to be positioned between said cylindrical seal member and the valve shaft and in fluid communication with air outside the valve assembly.

20. The valve component of claim 19, wherein said cylindrical seal member is configured to seal between the one or more bleed passages therethrough and a suction-source port defined in a valve well of the valve assembly.
